(19) United States
(12) Patent Application Publication
(10) Pub. No.: US 2021/0000407 A1
(43) Pub. Date: Jan. 7, 2021

(54) SYSTEMS AND METHODS FOR A BRAIN ACOUSTIC RESONANCE SEIZURE MONITOR

(71) Applicant: EpilepsyCo Inc., Guilford, CT (US)

(72) Inventors: Kamyar Firouzi, San Jose, CA (US); Eric Kabrams, Redwood City, CA (US); Mohammad Moghadamfalahi, San Jose, CA (US)

(73) Assignee: EpilepsyCo Inc., Guilford, CT (US)

(21) Appl. No.: 16/920,024

(22) Filed: Jul. 2, 2020

Related U.S. Application Data
(60) Provisional application No. 62/870,569, filed on Jul. 3, 2019.
(60) Provisional application No. 62/870,579, filed on Jul. 3, 2019.
(60) Provisional application No. 62/870,590, filed on Jul. 3, 2019.
(60) Provisional application No. 62/870,547, filed on Jul. 3, 2019.
(60) Provisional application No. 62/870,555, filed on Jul. 3, 2019.
(60) Provisional application No. 62/870,562, filed on Jul. 3, 2019.

(51) Int. Cl. A61B 5/00; A61B 8/08; A61B 8/00

(52) U.S. Cl. CPC A61B 5/4094; A61B 5/0022; A61B 5/0042; A61B 5/6803; A61B 5/031; A61B 5/7264; A61B 8/0808; A61B 8/4427; A61B 8/5207; A61B 5/6868

(57) ABSTRACT

In some aspects, the described systems and methods provide for a method comprising transmitting to a brain of a patient, with at least one transducer, acoustic signals. The method further comprises receiving from the brain, with the at least one transducer, data acquired from the brain including information related to standing waves, distribution of acoustic modes, frequency response, and/or impulse/transient response. The method further comprises detecting, from the acquired data, a seizure of the person.

### CROSS REFERENCE TO RELATED APPLICATIONS

This application claims priority under 35 U.S.C. § 119(e) to U.S. Provisional Application Ser. No. 62/870,569, titled “SYSTEMS AND METHODS FOR A BRAIN ACOUSTIC RESONANCE INTRACRANIAL PRESSURE MONITOR,” filed Jul. 3, 2019, U.S. Provisional Application Ser. No. 62/870,579, titled “SYSTEMS AND METHODS FOR A SKULL LAMB WAVES INTRACRANIAL PRESSURE MONITOR,” filed Jul. 3, 2019, U.S. Provisional Application Ser. No. 62/870,590, titled “SYSTEMS AND METHODS FOR A BRAIN ACOUSTIC RESONANCE SEIZURE MONITOR,” filed Jul. 3, 2019, U.S. Provisional Application Ser. No. 62/870,547, titled “SYSTEMS AND METHODS FOR TUMOR DETECTION,” filed Jul. 3, 2019, U.S. Provisional Application Ser. No. 62/870,555, titled “SYSTEMS AND METHODS FOR MAPPING DISTRIBUTION OF INTRACRANIAL PRESSURE,” filed Jul. 3, 2019, and U.S. Provisional Application Ser. No. 62/870,562, titled “SYSTEMS AND METHODS FOR SEIZURE LOCALIZATION,” filed Jul. 3, 2019, all of which are hereby incorporated herein by reference in their entireties.

### BACKGROUND

Neurological disorders affecting brain health constitute a significant portion of the global burden of disease. Such disorders can include epilepsy, Alzheimer's disease, and Parkinson's disease. For example, about 65 million people worldwide suffer from epilepsy. In the developing world, onset is more common in older children and young adults, due to differences in the frequency of the underlying causes. Nearly 80% of cases occur in the developing world. In the developed world, onset of new cases occurs most frequently in babies and the elderly. The United States itself has about 3.4 million people suffering from epilepsy with an estimated $15 billion economic impact. These patients suffer from symptoms such as recurrent seizures, which are episodes of excessive and synchronized neural activity in the brain. In many areas of the world, those with epilepsy either have restrictions placed on their ability to drive or are not permitted to drive until they are free of seizures for a specific length of time.

### SUMMARY

In some aspects, the methods/devices described herein provide for monitoring brain conditions as well as functions using direct acoustic sensing in a manner that is noninvasive (or minimally invasive), and in some cases, wireless, and continuous as well. In some embodiments, noninvasive sensors may be disposed or worn on the scalp or another suitable portion of the head. In some embodiments, minimally invasive sensors may be placed or implanted under the scalp or another suitable portion of the head. Acoustic or sound in a broad sense herein refers to any physical process that involves propagation of mechanical waves including, e.g., ultrasound and elastic waves. Brain functions, to be diagnosed and monitored, may include but are not limited to detection of epileptic seizure. Brain conditions, to be diagnosed and monitored, may include but are not limited to intracranial pressure, vasospasm, hemorrhage, and brain tumor. In some embodiments, sensors such as ultrasonic transducers, either standalone or in pairs, are utilized to send and receive acoustic waves into/from the brain with various form factors including, e.g., wearable as well as implantable devices. Through a pulsation protocol, the device may be capable of detecting changes in the brain that come from changes in functions or conditions of the brain. For example, changes may occur due to an elevated intracranial pressure (ICP) or prior to an epileptic seizure. These changes may be detected as mechanical changes in the form of steady pressure or low frequency tissue strain.

In some aspects, the described systems and methods provide for a method comprising transmitting to a brain of a patient, with at least one transducer, acoustic signals. The method further comprises receiving from the brain, with the at least one transducer, data acquired from the brain including information related to standing waves, distribution of acoustic modes, frequency response, and/or impulse/transient response. The method further comprises determining, from the acquired data, intracranial pressure of the person.

In some embodiments, determining the intracranial pressure includes assessing changes in amplitude, bandwidth, and/or frequency of the standing waves.

In some embodiments, the method further comprises transmitting, to an external device with a processor for determining the intracranial pressure, the acquired data.

In some embodiments, the at least one transducer includes a first transducer for transmitting the acoustic signals and receiving the acquired data.

In some embodiments, the at least one transducer includes a first transducer for transmitting the acoustic signals and a second transducer for receiving the acquired data.

In some aspects, the described systems and methods provide for a device comprising at least one transducer that transmits to the brain of a person acoustic signals and receives data acquired from the brain including information related to standing waves, distribution of acoustic modes, frequency response, and/or impulse/transient response for determining, based on the acquired data, intracranial pressure of the person.

In some embodiments, the device is wearable by the person.

In some embodiments, the device is implantable within a skull of the person.

In some embodiments, the device is portable.

In some embodiments, the at least one transducer includes a first transducer for transmitting the acoustic signals and receiving the acquired data.

In some embodiments, the at least one transducer includes a first transducer for transmitting the acoustic signals and a second transducer for receiving the acquired data.

In some aspects, the described systems and methods provide for at least one non-transitory computer-readable storage medium storing processor-executable instructions that, when executed by at least one computer hardware processor, cause the at least one computer hardware processor to perform the acts of transmitting to a brain of a patient, with at least one transducer, acoustic signals, receiving from the brain, with the at least one transducer, data acquired from the brain including information related to standing waves, distribution of acoustic modes, frequency response, and/or impulse/transient response, and determining, from the acquired data, intracranial pressure of the person.

In some embodiments, determining the intracranial pressure includes providing the acquired data to a machine learning model trained to predict the intracranial pressure.

In some embodiments, the at least one transducer includes a first transducer for transmitting the acoustic signals and receiving the acquired data.

In some embodiments, the at least one transducer includes a first transducer for transmitting the acoustic signals and a second transducer for receiving the acquired data.

In some aspects, the described systems and methods provide for a method comprising transmitting to a skull of a patient, with at least one transducer, acoustic signals. The method further comprises receiving from the skull, with the at least one transducer, data acquired from the skull including information related to guided waves, distribution of acoustic modes, frequency response, and/or impulse/transient response. The method further comprises determining, from the acquired data, intracranial pressure of the person.

In some embodiments, determining the intracranial pressure includes assessing changes in amplitude, bandwidth, and/or frequency of the guided waves.

In some embodiments, the method further comprises transmitting, to an external device with a processor for determining the intracranial pressure, the acquired data.

In some embodiments, the at least one transducer includes a first transducer for transmitting the acoustic signals and receiving the acquired data.

In some embodiments, the at least one transducer includes a first transducer for transmitting the acoustic signals and a second transducer for receiving the acquired data.

In some aspects, the described systems and methods provide for a device comprising at least one transducer that transmits to the skull of a person acoustic signals and receives data acquired from the skull including information related to guided waves, distribution of acoustic modes, frequency response, and/or impulse/transient response, for determining, based on the acquired data, intracranial pressure of the person.

In some embodiments, the device is wearable by the person.

In some embodiments, the device is implantable within the skull of the person.

In some embodiments, the device is portable.

In some embodiments, the at least one transducer includes a first transducer for transmitting the acoustic signals and receiving the acquired data.

In some embodiments, the at least one transducer includes a first transducer for transmitting the acoustic signals and a second transducer for receiving the acquired data.

In some aspects, the described systems and methods provide for at least one non-transitory computer-readable storage medium storing processor-executable instructions that, when executed by at least one computer hardware processor, cause the at least one computer hardware processor to perform the acts of transmitting to a skull of a patient, with at least one transducer, acoustic signals, receiving from the skull, with the at least one transducer, data acquired from the brain including information related to guided waves, distribution of acoustic modes, frequency response, and/or impulse/transient response, and determining, from the acquired data, intracranial pressure of the person.

In some embodiments, determining the intracranial pressure includes providing the acquired data to a machine learning model trained to predict the intracranial pressure.

In some embodiments, the at least one transducer includes a first transducer for transmitting the acoustic signals and receiving the acquired data.

In some embodiments, the at least one transducer includes a first transducer for transmitting the acoustic signals and a second transducer for receiving the acquired data.

In some aspects, the described systems and methods provide for a method comprising transmitting to a brain of a patient, with at least one transducer, acoustic signals. The method further comprises receiving from the brain, with the at least one transducer, data acquired from the brain including information related to standing waves, distribution of acoustic modes, frequency response, and/or impulse/transient response. The method further comprises detecting, from the acquired data, a seizure of the person.

In some embodiments, detecting the seizure includes assessing changes in amplitude, bandwidth, and/or frequency of the standing waves.

In some embodiments, the method further comprises transmitting, to an external device with a processor for detecting the seizure, the acquired data.

In some embodiments, the at least one transducer includes a first transducer for transmitting the acoustic signals and receiving the acquired data.

In some embodiments, the at least one transducer includes a first transducer for transmitting the acoustic signals and a second transducer for receiving the acquired data.

In some embodiments, determining a location of a seizure site based on the standing waves. In some aspects, the described systems and methods provide for a device comprising at least one transducer that transmits to the brain of a person acoustic signals and receives data acquired from the brain including information related to standing waves, distribution of acoustic modes, frequency response, and/or impulse/transient response, for detecting, based on the acquired data, a seizure of the person.

In some embodiments, the device is wearable by the person.

In some embodiments, the device is implantable within the skull of the person.

In some embodiments, the device is portable.

In some embodiments, the at least one transducer includes a first transducer for transmitting the acoustic signals and receiving the acquired data.

In some embodiments, the at least one transducer includes a first transducer for transmitting the acoustic signals and a second transducer for receiving the acquired data.

In some aspects, the described systems and methods provide for at least one non-transitory computer-readable storage medium storing processor-executable instructions that, when executed by at least one computer hardware processor, cause the at least one computer hardware processor to perform the acts of transmitting to a brain of a patient, with at least one transducer, acoustic signals, receiving, from the brain, with the at least one transducer, data acquired from the brain including information related to standing waves, distribution of acoustic modes, frequency response, and/or impulse/transient response, and detecting, from the acquired data, a seizure of the person.

In some embodiments, detecting the seizure includes providing the acquired data to a machine learning model trained to predict the seizure of the person.

In some embodiments, the at least one transducer includes a first transducer for transmitting the acoustic signals and receiving the acquired data.

In some embodiments, the at least one transducer includes a first transducer for transmitting the acoustic signals and a second transducer for receiving the acquired data.

In some aspects, the described systems and methods provide for a method comprising transmitting to a brain and/or skull of a patient, with at least one transducer, acoustic signals. The method further comprises receiving from the brain and/or skull, with the at least one transducer, data acquired from the brain and/or skull including information related to standing waves, guided waves, distribution of acoustic modes, frequency response, and/or impulse/transient response. The method further comprises determining, from the acquired data, presence of a tumor within the brain of the person.

In some embodiments, the method further comprises determining a location of the tumor based on the acquired data.

In some embodiments, determining the presence of the tumor includes assessing changes in amplitude, bandwidth, and/or frequency of the standing waves and/or guided waves.

In some embodiments, the method further comprises transmitting, to an external device with a processor for determining the presence of the tumor, the acquired data.

In some embodiments, the at least one transducer includes a first transducer for transmitting the acoustic signals and receiving the acquired data.

In some embodiments, the at least one transducer includes a first transducer for transmitting the acoustic signals and a second transducer for receiving the acquired data.

In some aspects, the described systems and methods provide for at least one transducer that transmits to the brain and/or skull of a person acoustic signals and receives data acquired from the brain and/or skull including information related to standing waves, guided waves, distribution of acoustic modes, frequency response, and/or impulse/transient response, for determining, based on the acquired data, presence of a tumor within the brain of the person.

In some embodiments, the device is wearable by the person.

In some embodiments, the device is implantable within the skull of the person.

In some embodiments, the device is portable.

In some embodiments, the at least one transducer includes a first transducer for transmitting the acoustic signals and receiving the acquired data.

In some embodiments, the at least one transducer includes a first transducer for transmitting the acoustic signals and a second transducer for receiving the acquired data.

In some aspects, the described systems and methods provide for at least one non-transitory computer-readable storage medium storing processor-executable instructions that, when executed by at least one computer hardware processor, cause the at least one computer hardware processor to perform the acts of transmitting to a brain and/or skull of a patient, with at least one transducer, acoustic signals, receiving, from the brain and/or skull, with the at least one transducer, data acquired from the brain and/or skull including information related to standing waves, guided waves, distribution of acoustic modes, frequency response, and/or impulse/transient response, and determining, from the acquired data, presence of a tumor within the brain of the person.

In some embodiments, determining the presence of the tumor includes providing the acquired data to a machine learning model trained to predict the presence of a tumor within the brain of the person.

In some embodiments, the at least one transducer includes a first transducer for transmitting the acoustic signals and receiving the acquired data.

In some embodiments, the at least one transducer includes a first transducer for transmitting the acoustic signals and a second transducer for receiving the acquired data.

In some aspects, the described systems and methods provide for a method comprising transmitting, with at least one transducer, acoustic signals to a brain of a patient, wherein the at least one transducer is configured to induce excitation of a plurality of acoustic modes. The method further comprises receiving, with the at least one transducer, data acquired from the brain including information related to standing waves, frequency response, impulse/transient response, and/or distribution of acoustic modes. The method further comprises determining, from the acquired data, a distribution of intracranial pressure within the brain of the person.

In some embodiments, determining the distribution of intracranial pressure includes providing the acquired data to a machine learning model trained to predict the distribution of intracranial pressure.

In some embodiments, the method further comprises transmitting, to an external device with a processor for determining the distribution of intracranial pressure, the acquired data.

In some embodiments, the at least one transducer includes a first transducer for transmitting the acoustic signals and receiving the acquired data.

In some embodiments, the at least one transducer includes a first transducer for transmitting the acoustic signals and a second transducer for receiving the acquired data.

In some aspects, the described systems and methods provide for a device comprising at least one transducer that transmits acoustic signals to the brain of a person and receives data acquired from the brain including information related to standing waves, frequency response, impulse/transient response, and/or distribution of acoustic modes, for determining, based on the acquired data, a distribution of intracranial pressure.

In some embodiments, the device is wearable by the person.

In some embodiments, the device is implantable within the skull of the person.

In some embodiments, the device is portable.

In some embodiments, the at least one transducer includes a first transducer for transmitting the acoustic signals and receiving the acquired data.

In some embodiments, the at least one transducer includes a first transducer for transmitting the acoustic signals and a second transducer for receiving the acquired data.

In some aspects, the described systems and methods provide for at least one non-transitory computer-readable storage medium storing processor-executable instructions that, when executed by at least one computer hardware processor, cause the at least one computer hardware processor to perform the acts of transmitting, with at least one transducer, acoustic signals to a brain of a patient, wherein the at least one transducer is configured to induce excitation of a plurality of acoustic modes, receiving, with the at least one transducer, data acquired from the brain including information related to standing waves, frequency response, impulse/transient response, and/or distribution of acoustic modes, and determining, from the acquired data, a distribution of intracranial pressure within the brain of the person.

In some embodiments, determining the distribution of intracranial pressure includes providing the acquired data to a machine learning model trained to predict the distribution of intracranial pressure.

In some embodiments, the at least one transducer includes a first transducer for transmitting the acoustic signals and receiving the acquired data.

In some embodiments, the at least one transducer includes a first transducer for transmitting the acoustic signals and a second transducer for receiving the acquired data.

In some aspects, the described systems and methods provide for a method comprising transmitting, with at least one transducer, acoustic signals to a brain of a patient, wherein the at least one transducer is configured to induce excitation of a plurality of acoustic modes. The method further comprises receiving, with the at least one transducer, data acquired from the brain including information related to standing waves, frequency response, impulse/transient response, and/or distribution of acoustic modes. The method further comprises determining, from the acquired data, a location of a seizure site within the brain of the person.

In some embodiments, determining the location of the seizure site includes providing the acquired data to a machine learning model trained to predict the location of the seizure site.

In some embodiments, the method further comprises transmitting, to an external device with a processor for determining the location of the seizure site, the acquired data.

In some embodiments, the at least one transducer includes a first transducer for transmitting the acoustic signals and receiving the acquired data.

In some embodiments, the at least one transducer includes a first transducer for transmitting the acoustic signals and a second transducer for receiving the acquired data.

In some aspects, the described systems and methods provide for a device comprising at least one transducer that transmits acoustic signals to the brain of a person and receives data acquired from the brain including information related to standing waves, frequency response, impulse/transient response, and/or distribution of acoustic modes, for determining, based on the acquired data, a location of a seizure site.

In some embodiments, the device is wearable by the person.

In some embodiments, the device is implantable within the skull of the person.

In some embodiments, the device is portable.

In some embodiments, the at least one transducer includes a first transducer for transmitting the acoustic signals and receiving the acquired data.

In some embodiments, the at least one transducer includes a first transducer for transmitting the acoustic signals and a second transducer for receiving the acquired data.

In some aspects, the described systems and methods provide for at least one non-transitory computer-readable storage medium storing processor-executable instructions that, when executed by at least one computer hardware processor, cause the at least one computer hardware processor to perform the acts of transmitting, with at least one transducer, acoustic signals to a brain of a patient, wherein the at least one transducer is configured to induce excitation of a plurality of acoustic modes, receiving, with the at least one transducer, data acquired from the brain including information related to standing waves, frequency response, impulse/transient response, and/or distribution of acoustic modes, and determining, from the acquired data, a location of a seizure site within the brain of the person.

In some embodiments, determining the location of the seizure site includes providing the acquired data to a machine learning model trained to predict the location of the seizure site.

In some embodiments, the at least one transducer includes a first transducer for transmitting the acoustic signals and receiving the acquired data.

In some embodiments, the at least one transducer includes a first transducer for transmitting the acoustic signals and a second transducer for receiving the acquired data.

While some aspects and/or embodiments described herein are described with respect to intracranial pressure or epilepsy-related applications, these aspects and/or embodiments may be equally applicable to monitoring and/or treating symptoms for any suitable neurological disorder. Any limitations of the embodiments described herein are limitations only of those embodiments, and are not limitations of any other embodiments described herein.

### BRIEF DESCRIPTION OF THE DRAWINGS

Various aspects and embodiments will be described with reference to the following figures. The figures are not necessarily drawn to scale.

FIG. 1 shows an illustrative example of a Brain Acoustic Resonance (BAR) intracranial pressure (ICP) monitor, in accordance with some embodiments of the technology described herein.

FIG. 2 shows a block diagram of an illustrative ICP monitor, in accordance with some embodiments of the technology described herein.

FIGS. 3A-3D shows an illustration of a Capacitive Micromachined Ultrasonic Transducer (CMUT) cell, in accordance with some embodiments of the technology described herein.

FIG. 4 shows an illustration of superposition and mode conversion of partial longitudinal and shear vertical waves along an isotropic elastic waveguide, in accordance with some embodiments of the technology described herein.

FIGS. 5A-5B show illustrative dispersion curves and mode-shapes for Lamb waves, in accordance with some embodiments of the technology described herein.

FIG. 6 shows an illustrative example of a skull Lamb waves ICP monitor, in accordance with some embodiments of the technology described herein.

FIG. 7 shows an illustrative example of a BAR seizure monitor, in accordance with some embodiments of the technology described herein.

FIG. 8 shows an overview of an illustrative algorithm for determining the intracranial pressure across the brain, its distribution, presence of seizure, location of seizure site, or other indicators of brain functions or conditions, in accordance with some embodiments of the technology described herein.

FIG. 9 shows an illustrative flow diagram for a process for constructing and deploying an algorithm, e.g., as shown in FIG. 8, in accordance with some embodiments of the technology described herein.

FIG. 10 shows exemplary input data for BAR-based ICP estimation, in accordance with some embodiments of the technology described herein.

FIG. 11 shows examples of acoustic resonances in the skull at a selection of frequencies, in accordance with some embodiments of the technology described herein.

FIG. 12 shows an example of the response of a sensor over the skull at different intracranial pressures and frequencies, in accordance with some embodiments of the technology described herein.

FIG. 13 shows an illustrative flow diagram for a process for ICP estimation, in accordance with some embodiments of the technology described herein.

FIG. 14 shows performance of a statistical model on test data via a confusion matrix, in accordance with some embodiments of the technology described herein.

FIG. 15 shows a convolutional neural network that may be used to detect and/or predict one or more symptoms of a neurological disorder, in accordance with some embodiments of the technology described herein.

FIG. 16 shows another convolutional neural network that may be used to detect and/or predict one or more symptoms of a neurological disorder, in accordance with some embodiments of the technology described herein.

FIG. 17 shows a block diagram of an illustrative computer system that may be used in implementing some embodiments of the technology described herein.

### DETAILED DESCRIPTION

The inventors herein have discovered that by transmitting and receiving ultrasound waves to the brain and/or skull, brain functions and conditions such as, epileptic seizure, intracranial pressure, vasospasm, hemorrhage, and brain tumor, can be diagnosed and treated noninvasively or minimally invasively. In some aspects, devices, methods, and systems described herein provide for monitoring brain conditions and functions non-invasively or minimally invasively. Such devices, methods and systems in some embodiments also diagnose and/or treat brain conditions. In some embodiments, noninvasive sensors may be disposed or worn on the scalp or another suitable portion of the head. In some embodiments, minimally invasive sensors may be placed or implanted under the scalp or another suitable portion of the head.

For example, the described systems and methods may be used to treat epilepsy, which is a group of neurological disorders characterized by epileptic seizures. Epileptic seizures are episodes that can vary from brief and nearly undetectable periods to long periods of vigorous shaking. These episodes can result in physical injuries, including occasionally broken bones. In epilepsy, seizures tend to recur and have no immediate underlying cause. Some cases occur as the result of brain injury, stroke, brain tumors, infections of the brain, and birth defects through a process known as epileptogenesis. In such cases, epileptic seizures are the result of excessive and abnormal brain function, including abnormal neuronal activity in the cortex of the brain. The diagnosis involves ruling out other conditions that might cause similar symptoms, such as fainting, and determining if another cause of seizures is present, such as alcohol withdrawal or electrolyte problems. This may be partly done by imaging the brain and performing blood tests. The diagnosis of epilepsy is typically made based on observation of the seizure onset and the underlying cause. A functional neuroimaging method, such as Electroencephalography (EEG), to look for abnormal patterns of brain function, including brain waves, and a structural neuroimaging method, such as Computed Tomography (CT) or Magnetic Resonance Imaging (MRI), to look at the structure of the brain are also usually part of the diagnosis. Epilepsy usually cannot be cured, unless surgery is performed. Common procedures include cutting out the hippocampus via an anterior temporal lobe resection, removal of tumors, and removing parts of the neocortex. Some procedures such as a corpus callosotomy may be attempted in an effort to decrease the number of seizures rather than cure the condition. However, the outcome of surgery can lead to unexpected harsh outcomes such as loss of functionality of certain abilities such as speech, control over movements, etc. Neurostimulation may be another option in those who are not candidates for surgery. Certain types of neurostimulation may be effective in those who do not respond to medications, including vagus nerve stimulation, anterior thalamic stimulation, and closed-loop responsive stimulation.

In some embodiments, brain functions and conditions, to be diagnosed and monitored using the described systems and methods, may include detection and monitoring of brain tumors and hemorrhage, including assessment of the clot volume and recurrence over time, assessment of the midline shift and brain compression, and guided insertion of catheters. Further, vasospasm detection and monitoring may be performed using the described systems and methods. Conventionally, bedside transcranial ultrasound may be used for vasospasm detection. However, passing of ultrasound waves through the skull may still be a limiting factor. Moreover, vasospasm can happen at any moment and patients are often critically ill and in coma without a reliable physical examination.

The inventors herein have recognized limitations with existing methods, systems, and devices for monitoring and treating brain function. Conventional non-invasive technology includes EEG and MRI/CT scans. EEG is an electrophysiological monitoring method to record electrical activity of the brain. EEG is typically noninvasive, with the electrodes placed along the scalp, although invasive electrodes are sometimes used such as in electrocorticography. EEG measures voltage fluctuations resulting from ionic current within the neurons of the brain. EEG is most often used to diagnose epilepsy, which causes abnormalities in EEG readings. EEG has poor spatial resolution for diagnosis. Often for proper diagnosis or detection of epilepsy both high temporal resolution and spatial resolution is required. To capture the structure of the nervous system and the diagnosis of large scale intracranial disease (such as tumor) or injury, and for detection of epileptic events, MRI and CT can be used. They provide good spatial resolution for diagnosis. However, they have poor temporal resolution. Moreover, they are very expensive and not portable. Despite limited spatial resolution, EEG is one of the few portable techniques available and offers millisecond-range temporal resolution which is not possible with CT or MRI. Moreover, early detection of disease recurrence and infra clinic brain changes requires a continuous imaging and/or monitoring system, which is especially important for early recurrence detection (e.g., for brain tumors).

In some aspects, the described systems and methods provide for a novel wearable or implantable intracranial pressure (ICP) monitoring unit capable of measuring the intracranial pressure, seizure, and/or other suitable conditions of a person by collectively exciting and receiving resonance modes of the skull and/or the brain. The inventors have appreciated that the skull and the brain as one entity form a resonant cavity which may consist of an infinite number of resonance modes. Intracranial pressure in the head of the person may appear as a small change in the mechanical properties of the brain and skull, which may manifest in the form of changes in the speed of sound or attenuation of acoustic waves. The frequency of the resonances, their amplitudes, phases, and/or bandwidth (or quality factor) may be functions of the speed of sound and/or attenuation, and thus, provide a means of measuring the intracranial pressure or any changes in tissue structure. In some embodiments, the measurement is conducted by pulsing an acoustic transducer (e.g., a device that converts electrical energy to mechanical energy, and vice versa) and listening to (e.g., measuring) the waves propagating in the skull, brain, or both, at the same transducer and/or other transducers worn or implanted on the head of the person. The resonances may be identified either in the time-domain by exciting the transducer via a short-duration pulse or in the frequency domain by exciting the transducer via a long single-frequency tone-burst repeated at various frequencies.

In some aspects, the described systems and methods provide for real-time, patient-specific and/or direct reading of ICP or seizure of the person using a wearable (or implantable) that is wireless, low power, miniaturized and/or AI (Artificial Intelligence)-powered.

Intracranial pressure (ICP) is defined as the pressure inside the skull, and therefore, the pressure inside the brain tissue and the cerebrospinal fluid (CSF). Brain tissue is a soft matter with hyper-elastic incompressible material behavior; it can experience large reversible deformation (or strain) while maintaining a constant total volume. Inside the brain, the relationship between CSF and intracranial blood volume is described by the Monroe Kellie doctrine, which states that because the brain is incompressible, when the skull is intact, the sum of the volumes of brain, CSF, and intracranial blood is constant. Incompressibility leads to the build-up of the background steady stress or pressure inside the brain. Changes in ICP acts as a steady stress which affects the based acoustic properties of the brain or skull. ICP is typically considered to be normal when within the range of 5-15 mmHg in a healthy supine adult, 3-7 mmHg in children, and 1.5-6 mmHg in infants. ICP may be considered to be elevated when higher than 20 mmHg. This may be considered as an important cause of secondary injury leading to irreversible brain injury and death.

The acoustoelastic effect relates to how the sound velocities (both longitudinal and shear wave velocities) of an elastic material change if subjected to an initial static stress field. This is a non-linear effect of the constitutive relation between mechanical stress and finite strain in a material of continuous mass. In classical linear elasticity theory, small deformations of most elastic materials can be described by a linear relation between the applied stress and the resulting strain. This relationship is commonly known as the generalized Hooke's law. The linear elastic theory involves second order elastic constants (known as Lame parameters) and yields constant longitudinal and shear sound velocities in an elastic material, not affected by an applied stress. The acoustoelastic effect on the other hand include higher order expansion of the constitutive relation (non-linear elasticity theory) between the applied stress and resulting strain, which yields longitudinal and shear sound velocities dependent of the stress state of the material. In the limit of an unstressed material the sound velocities of the linear elastic theory are reproduced.

ICP monitoring may be used for a number of conditions, e.g., traumatic brain injury, intracerebral hemorrhage, subarachnoid hemorrhage, hydrocephalus, malignant infarction, cerebral edema, CNS infections, hepatic encephalopathy etc. In these conditions, ICP monitoring in the light of other parameters may help influence management of the condition for better outcomes. For some conditions it may be important to monitor ICP as even minor fluctuations may require a change in management. Conventionally, ICP may be monitored using an invasive intraventricular catheter connected to an external pressure transducer. For example, the catheter may be placed into one of the ventricles through a burr hole. The catheter can also be used for therapeutic CSF drainage and for administration of drugs. Even though this conventional method may be an accurate and cost-effective method of ICP monitoring, it is associated with a number of complications. These include risk of infection, hemorrhage, obstruction, difficulty in placement, malposition, etc. Other invasive modalities for ICP monitoring, all of which entail the same complications as intraventricular catheter insertion, include intraparenchymal monitors, subdural, and epidural devices, as well as lumbar puncture measurements.

Complications of invasive ICP monitoring may include disconnection, device failure, infection, and hemorrhage. Ventricular-catheter related infection rates are around 10% and are associated with the duration of catheter placement. The use of antibiotic impregnated catheters can potentially reduce the risk of infection by prolonging the mean duration to onset of infection. Clinically symptomatic hemorrhages due to the catheter range from 0.7% to 2.4%. Conventional technologies include transcranial ultrasound doppler, Near Infrared Spectroscopy, MRI, CT, EEG, etc. More information can be found in M. N. Khan et al., “Noninvasive monitoring intracranial pressure: A review of available modalities,” Surg Neurol Int. 2017; 8: 51, April 2017, which is incorporated herein by reference in its entirety.

In some embodiments, the devices described herein include a resonator that exhibits resonance or resonant behavior. That is, it naturally oscillates with greater amplitude at some frequencies, called resonant frequencies, than at other frequencies. The oscillations in a resonator can be either electromagnetic or mechanical (including acoustic). Resonators are used to either generate waves of specific frequencies or to select specific frequencies from a signal. Musical instruments use acoustic resonators that produce sound waves of specific tones. Another example is quartz crystals used in electronic devices such as radio transmitters and quartz watches to produce oscillations of very precise frequency. A cavity resonator is one in which waves exist in an isolated or bounded space inside the device. Examples of acoustic cavity resonators include guitar string or a Helmholtz resonator (in which sound is produced by air vibrating in a cavity with one opening).

The key properties of the resonances are the frequency of resonance, amplitude, phase, Q-factor (or equivalently fractional bandwidth). Q-factor (or quality factor) is a dimensionless parameter that describes how underdamped an oscillator or resonator is and characterizes a resonator's bandwidth relative to its center frequency. Higher Q indicates a lower rate of energy loss relative to the stored energy of the resonator; the oscillations die out more slowly. A pendulum suspended from a high-quality bearing, oscillating in air, has a high Q, while a pendulum immersed in oil has a low one. Resonators with high quality factors have low damping, so that they ring or vibrate longer. As such high Q resonators can be perturbed much more easily than the low Q one.

In some embodiments, the devices described herein include ultrasonic transducers, either standalone or in pairs, which are utilized to send and receive acoustic waves into/from the brain with various form factors including, e.g., wearable as well as implantable. Through a pulsation protocol, the device may be capable of detecting changes in the brain that come from changes in functions or conditions of the brain. For example, changes may occur due to an elevated intracranial pressure (ICP) or prior to an epileptic seizure. These are mechanical changes in the form of steady pressure or low frequency tissue strain.

In some embodiments, the devices described herein can be either wearable or implantable (e.g., under the scalp). In the wearable form, the form factor for the devices can be one or several small adhesive patches. Alternatively, the devices can be integrated into a helmet or cap. The devices can be wirelessly charged and transfer data to a hub that can be worn (such as a watch or smart phone) or implanted (such as a small patch over the neck/arm).

Brain Acoustic Resonance (BAR) Intracranial Pressure Monitor

In some embodiments, the described systems and methods provide for a BAR intracranial pressure monitor to excite and listen to the acoustic modes in the head (e.g., brain and skull together) by putting a small wearable or implantable transducer over the head. Acoustic modes may be defined as mechanical vibrations at certain natural frequencies, where the acoustic system, e.g., the human head, experiences a larger magnitude of vibration when the frequency of excitation matches one of the natural frequencies. The transducer is very small (e.g., on the order of 1-2 centimeters or another suitable size) and may provide high spatial bandwidth to excite as many modes as possible (e.g., on the order of tens of modes or another suitable number). In some embodiments, only one transducer may be sufficient to measure ICP. In some embodiments, for having more local readings, more transducers can be populated over the head.

Wave propagation in complex structures in the high frequency (and small wavelength) limit is complex and rich in information. For example, for a skull that is 15 centimeters wide in lateral directions, any frequencies on the order of tens of kilohertz or more (e.g., wavelengths on the order of 1-2 centimeters or less) may fall within this section. The complexity of wave media can be either due to the presence of subwavelength inhomogeneity (e.g., due to scattering objects), or to the geometrical boundaries enclosing a homogeneous medium. Complexity due to geometrical boundaries may be addressed using quantum chaos theory to describe a high energy state (e.g., the analogue of Eigen-frequencies in an acoustic enclosure) solutions of the Schrodinger equation. Because the Helmholtz equation is the formal analogue of the Schrodinger equation for electromagnetic and acoustic waves, the field of wave chaos has emerged accordingly. Acoustic enclosures are common examples of wave cavities where the dynamics of rays may display chaos. Wave chaos leads to rich wave phenomena such as universal statistical behaviors of the frequency spectra and certain spatial patterns of the modes of the corresponding enclosure. The statistical behavior of the high frequency modes may be dependent upon the geometry of the enclosure.

In a classical setting, there may be two types of motion: regular (or integrable) and irregular (or chaotic). Regular domains have stable trajectories and may also exhibit caustics, e.g., regions that the ray trajectories never visit regardless of the number of reflections. In chaotic domains, on the other hand, the trajectories are unstable and ergodic, meaning they interrogate all the points in the wave domain almost surely. These modes exhibit sensitive dependence on the initial conditions/inputs. Instability of the ray trajectories in wave enclosures is the manifestation of extreme sensitivity to the inputs of the system. Furthermore, the geometry of regular systems can be sensitive to any perturbation to the geometry so that any irregular perturbation in the order of a wavelength can turn it into a chaotic domain. These modes of a bounded wave domain can be speckle-like (e.g., ergodic) or scarred. A scarred mode may be realized through intensity enhancement in a vicinity of a subset of the wave domain. An ergodic behavior motivates that information will reach out everywhere with equal probability, whereas a scarred behavior implies information is mainly trapped over a sub region of the domain. When an object is placed in an otherwise homogeneous regular domain, it effectively perturbs the base wave properties such as the refractive index, which in turn perturbs some of the base modes by ergodic ones.

Reverberation is another aspect of the complexity of waves in enclosures. It is the process of formation of a wave field in enclosures as a result of a large number of reflections. It leads to mixing of the wave energy, which in turn results in incoherent spreading of information. Reverberation is generally identified by the transient behavior of wave fields in enclosures. If one considers rays as trajectories of point-like particles carrying the wave energy, then the energy flow would exhibit a uniform isotropic distribution in chaotic domains. In contrast to optics, where all wavelengths are generally very short with respect to objects, in acoustics/ultrasound, numerous length-scales coexist, suggesting that the diffraction effects and complicated scattering patterns are of equal importance and must be considered. Reverberation can be understood as a random superposition of acoustic modes of the cavity.

The inventors have appreciated that the human head (e.g., the skull and everything inside) is a chaotic resonant acoustic cavity. Through a collective excitation of all acoustic modes, by setting up a reverberant field, it may be ensured that the ergodic modes are excited and interrogate all points in the brain. This in turn may ensure perturbations anywhere in the brain will affect some or all of these modes. At the frequency range of interest, the attenuation of sound waves is very low; thus, the modes are high-Q modes and very sensitive to any perturbations. Field reverberation helps to excite and monitor the perturbation as a function of time. It also makes sure maximum amount of information of perturbations are registered. As such it helps with mapping all the spatial information onto time and thus reducing the number of spatial measurements.

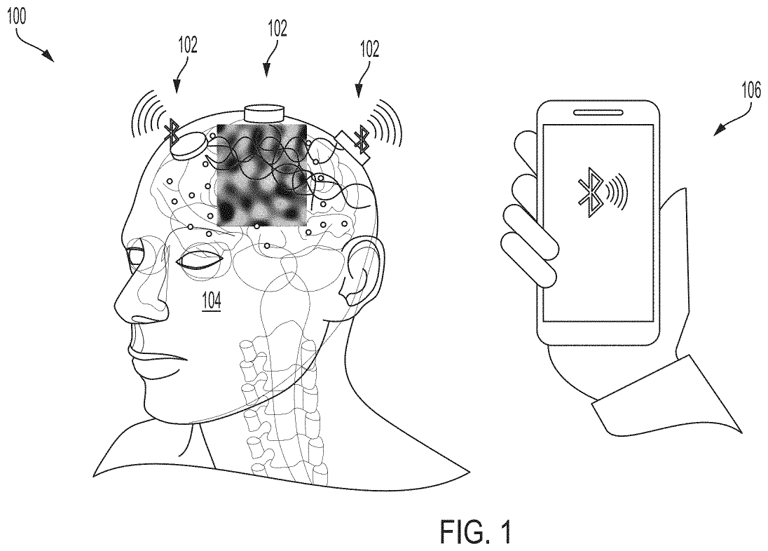

FIG. 1 shows an illustrative example 100 of a Brain Acoustic Resonance (BAR) intracranial pressure (ICP) monitor, in accordance with some embodiments of the technology described herein. In FIG. 1, acoustic transducers 102 may set up a reverberant field (also known as chaotic standing waves) in the brain of a person 104. The standing waves are a superposition of acoustic resonances of the brain and are modulated at different intracranial pressures. In the superimposed image, the sine wave-like traces indicate the waves going from one acoustic transducer 102 to another. The image shows a reverberant standing wave pattern that is set up as a result of multiple reflections of the waves in the skull. In some embodiments, the peak amplitude of a standing wave's oscillations at any point in space may be constant with time, and the oscillations at different points throughout the standing wave may be in phase. The received waveform may be wirelessly transmitted to a hub 106 like APPLE WATCH or IPHONE or another suitable device via BLUETOOTH or another suitable communication means.

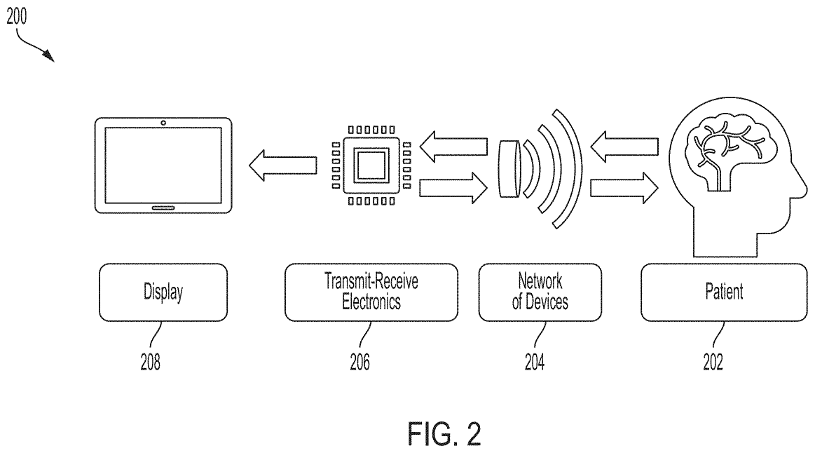

FIG. 2 shows a block diagram 200 of an illustrative ICP monitor, in accordance with some embodiments of the technology described herein. Patient 202 may have a network of devices 204, e.g., acoustics transducers 102, disposed on his or her head. The network of devices 204 may use transmit-receive electronics 206 to transmit data, e.g., e.g., wirelessly, BLUETOOTH or another suitable communication means, acquired from the brain and/or skill of patient 202. This data may be processed and/or displayed at display 208. For example, the data may include a waveform received from the patient's head at an APPLE WATCH or IPHONE or another suitable device that includes display 208.

In some embodiments, a method, a system, and/or a device for a brain acoustic resonance intracranial pressure monitor transmits acoustic signals to the brain using one or more transducers. The transducers receives data acquired from the brain, including information related to standing waves, distribution of acoustic modes, frequency response, and/or impulse/transient response. The frequency response may represent a quantitative measure of the output from the brain in response to a signal. For example, the frequency response may include a measure of magnitude and phase of the output as a function of frequency, in comparison to the input. While the impulse response may represent the response from the brain when presented with a brief input signal, the transient response may represent the response from the brain when changing from an equilibrium or a steady state. In some embodiments, the same transducer(s) transmit the acoustic signals to the brain and receive the data acquired from the brain. In some embodiments, the transducer(s) used for transmitting the acoustic signals to the brain are different from the transducer(s) used for receiving the data acquired from the brain.

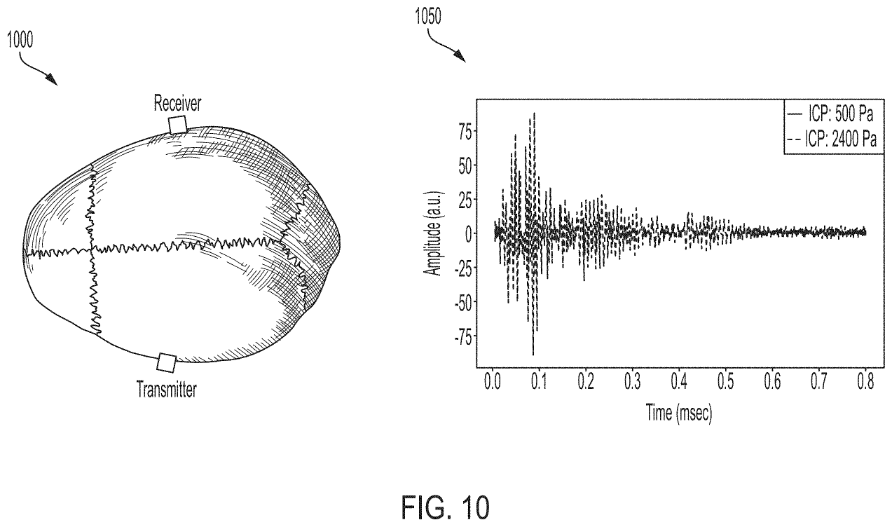

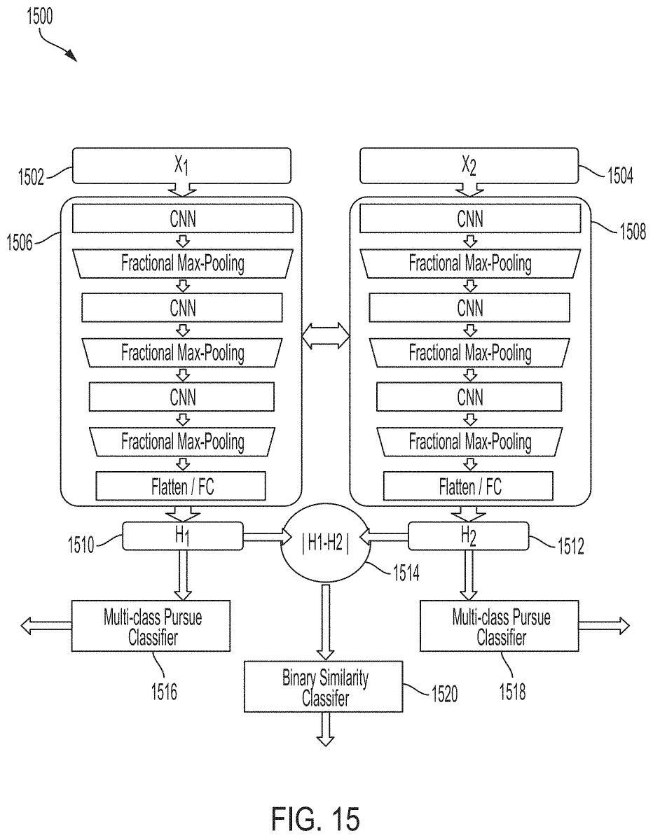

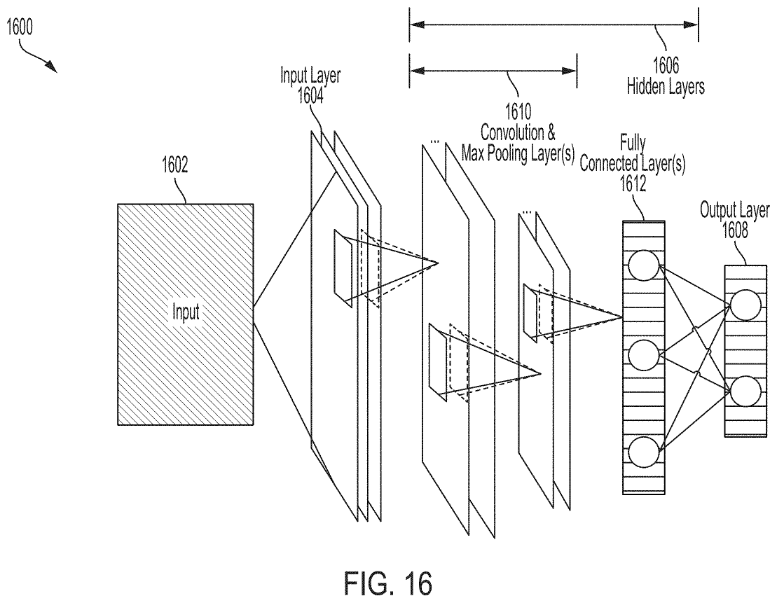

The intracranial pressure is determined from the acquired data, e.g., as shown in FIG. 10. For example, determining the intracranial pressure may include assessing changes in amplitude, bandwidth, and/or frequency of the standing waves. Additionally or alternatively, the acquired data may be transmitted to an external device with a processor to determine the intracranial pressure. For example, the intracranial pressure may be determined at the external device using a statistical model (e.g., as described with respect to FIGS. 15-16 or another suitable statistical model) that receives at least a portion of the acquired data as input and outputs a measure of intracranial pressure or related information suitable for determining the intracranial pressure. The device for the brain acoustic resonance intracranial pressure monitor may be wearable by the person, implantable within a skull of the person, and/or portable in nature.

In some embodiments, techniques for exciting the modes of the skull and/or brain described throughout this disclosure may include direct-surface bonded transducers, wedge transducers, and/or interdigital transducers/comb transducers. Transducers can be of a variety of types such as Piezoelectric, CMUT (Capacitive Micromachined Ultrasonic Transducer), Electro Magnetic Acoustic Transducer (EMAT), Piezoelectric Micromachined Ultrasonic Transducer (PMUT), etc. Material and dimensions determine the bandwidth and sensitivity of the transducer. CMUTs are of particular interest compared to other types of transducers as they can be easily miniaturized even at low frequencies, have superior sensitivity as well as wide bandwidth.

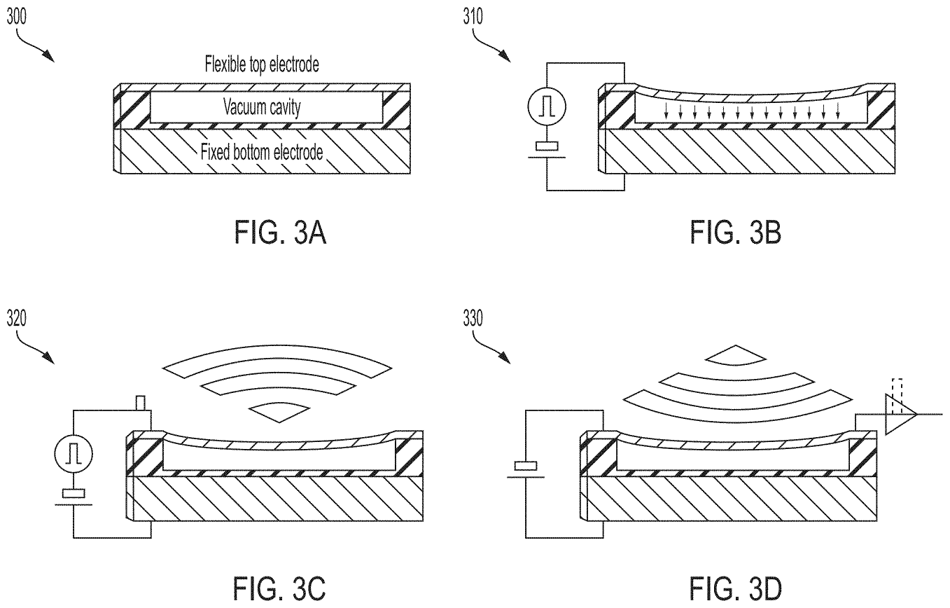

In some embodiments, the CMUT includes a flexible top plate suspended over a gap, forming a variable capacitor. The displacement of the top plate creates an acoustic pressure in the medium (or vice versa; acoustic pressure In the medium displaces the flexible plate). Transduction is achieved electrostatically, by converting the displacement of the plate to an electric current through modulating the electric field in the gap, in contrast with piezoelectric transducers. The merit of the CMUT derives from having a very large electric field in the cavity of the capacitor, a field of the order of 10\hat{ }8 V/m or higher results in an electro-mechanical coupling coefficient that competes with the best piezoelectric materials. The availability of micro-electro-mechanical-systems (MEMS) technologies makes it possible to realize thin vacuum gaps where such high electric fields can be established with relatively low voltages. Thus, viable devices can be realized and even integrated directly on electronic circuits such as complimentary metal-oxide-semiconductor (CMOS). FIGS. 3A-3D shows illustrations 300, 310, 320, and 330 of a CMUT cell (a) without DC bias voltage (FIG. 3A), and (b) with DC bias voltage (FIG. 3B), and principle of operation during (c) transmit (FIG. 3C) and (d) receive (FIG. 3D).

In some embodiments, a further aspect is collapse mode operation of the CMUT. In this mode of operation, the CMUT cells are designed so that part of the top plate is in physical contact with the substrate, yet electrically isolated with a dielectric, during normal operation. The transmit and receive sensitivities of the CMUT are further enhanced thus providing a superior solution for ultrasound transducers. In short, the CMUT is a high electric field device, and if one can control the high electric field from issues like charging and breakdown, then one has an ultrasound transducer with superior bandwidth and sensitivity, amenable for integration with electronics, manufactured using traditional integrated circuits fabrication technologies with all its advantages, and can be made flexible for wrapping around a cylinder or even over human tissue.

Skull Lamb Waves Intracranial Pressure Monitor

In some embodiments, the described systems and methods provide for a skull Lamb waves intracranial pressure monitor to excite and listen to the guided waves (also called Lamb waves) in the skull and monitor the behavior of these Lamb waves in response to changes in the brain conditions such as intracranial pressure. Guided waves in the skull adjacent to a fluid medium (such as liquid or gas) can leak, through mode-conversion from guided waves to compressional acoustic waves. Mode-converted compressional waves can also mode-convert back into guided waves through the reciprocity principle. The mode-conversion or leak rate is approximately a few wavelengths. When there is a change in the brain condition such as ICP, the mode-conversion or leak rate changes as a result. Moreover, ICP may lead to expansion of the skull because of its elasticity. Therefore, Lamb waves propagating between two fixed points over the skull travel different distances at different ICPs, yet providing another marker to measure and monitor ICP.

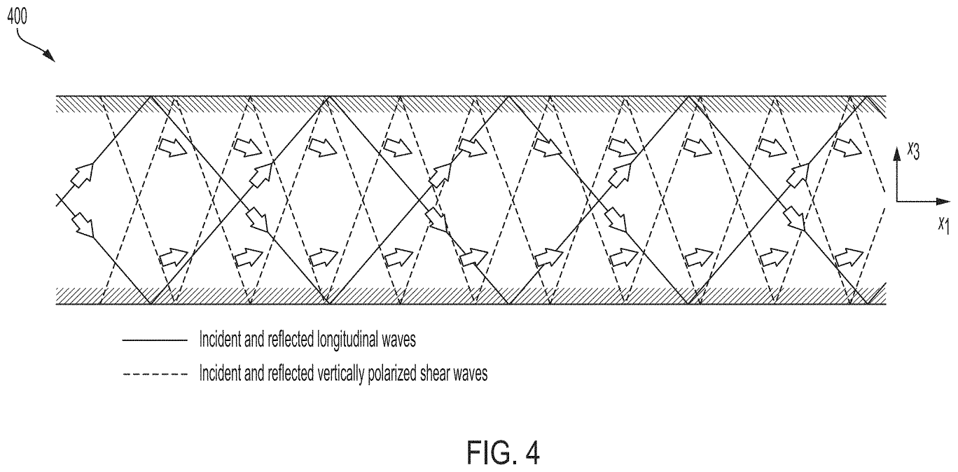

Rayleigh-Lamb waves (or Lamb waves) are guided elastic waves that propagate in bounded elastic media. The human skull bone is transversally thin, and thus effectively, appears as an elastic waveguide that can support propagation of Lamb waves. For thin structures such as plates, the compressional and shear waves do not exist independently, but are coupled. FIG. 4 shows an illustration 400 of superposition and mode conversion of partial longitudinal and shear vertical waves along an isotropic elastic waveguide. As the waves propagate, as shown in FIG. 4, both longitudinal and shear waves repeatedly bounce off the upper and lower boundaries, at which they mode-convert into one another. The superposition of these waves leads to a certain class of guided waves called Lamb waves which can propagate along bounded elastic media such as the skull bone. They can propagate without significant attenuation and can leak into the surrounding medium efficiently.

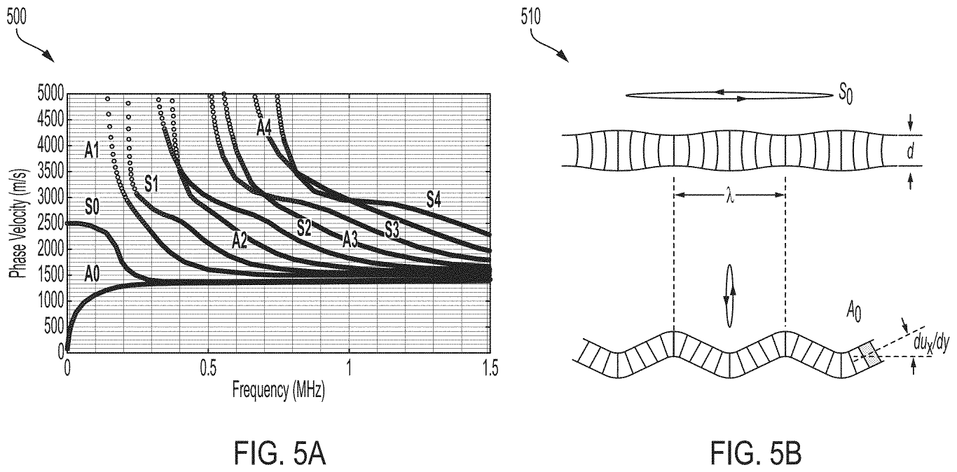

The inventors have appreciated feasibility of exciting and propagating Lamb waves in bone, e.g., for measuring ICP or another suitable application. Lamb waves come in different frequency dependent modes. The dispersion curves and some of the mode-shapes are shown in FIGS. 5A-5B. Dispersion is the dependence of the propagation velocity on the frequency. Dispersion is considered very weak in soft tissues and generally neglected. However, it has a strong effect on the propagation of Lamb waves. FIGS. 5A-5B show illustrations 500 and 510 of Lamb waves phase velocities as a function of frequency and the corresponding schematic of the modal deformation of the lowest order symmetric and asymmetric modes: (a) Lamb waves phase-velocities' dispersion curves (FIG. 5A), (b) S0 and A0 mode-shapes (FIG. 5B). As it can be seen by the examples of the mode-shape in FIG. 5B, Lamb waves couple the displacement of the upper and lower surfaces (outer and inner in the case of the skull), unlike the surface waves or bulk waves. Lamb waves put adjacent to an acoustic medium (such as water or soft tissue) can leak. The leak rate is approximately a few wavelengths.

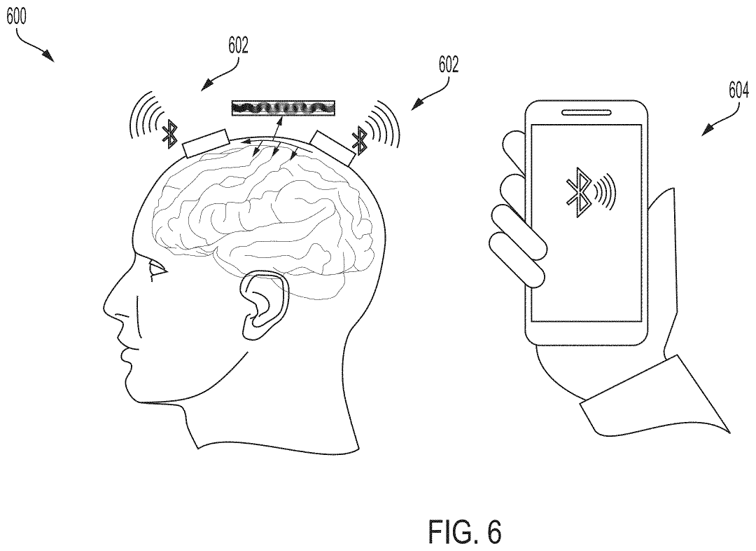

FIG. 6 shows an illustrative example 600 of a skull Lamb waves ICP monitor, in accordance with some embodiments of the technology described herein. In FIG. 6, Lamb waves propagating from the transmitters 602 may arrive at the receiver with different phases and amplitudes at different intracranial pressures. The received waveforms may be wirelessly transmitted to a hub 604 like APPLE WATCH or IPHONE or another suitable device via BLUETOOTH or another suitable communication means.

In some embodiments, a method, a system, and/or a device for a skull Lamb waves intracranial pressure monitor transmits acoustic signals to the skull using one or more transducers. The transducers receives data acquired from the skull, including information related to guided waves, distribution of acoustic modes, frequency response, and/or impulse/transient response. In some embodiments, the same transducer(s) transmit the acoustic signals to the brain and receive the data acquired from the brain. In some embodiments, the transducer(s) used for transmitting the acoustic signals to the brain are different from the transducer(s) used for receiving the data acquired from the brain.

The intracranial pressure is determined from the acquired data. For example, determining the intracranial pressure may include assessing changes in amplitude, bandwidth, and/or frequency of the guided waves. Additionally or alternatively, the acquired data may be transmitted to an external device with a processor to determine the intracranial pressure. For example, the intracranial pressure may be determined at the external device using a statistical model (e.g., as described with respect to FIGS. 15-16 or another suitable statistical model) that receives at least a portion of the acquired data as input and outputs a measure of intracranial pressure or related information suitable for determining the intracranial pressure. The device for the skull Lamb waves intracranial pressure monitor may be wearable by the person, implantable within a skull of the person, and/or portable in nature.

In some embodiments, at low frequencies, the wavelength is larger; thus, the penetration depth of the acoustic waves in the brain is larger. This is a suitable environment for the BAR intracranial pressure monitor described herein, which attempts to estimate the overall pressure in the brain. At high frequencies, the penetration depth becomes smaller, which is more suited for the skull Lamb waves intracranial pressure monitor and can provide a local reading of the ICP. For example, in a hospital surgery setting, ICP may be locally measured using the skull Lamb waves intracranial pressure monitor to determine where to drill into the person's skull. In another example, in an emergency room setting, ICP may be measured as a whole using the BAR intracranial pressure to determine entire brain health for the person.

Brain Acoustic Resonance (BAR) Seizure Monitor

In some embodiments, the described systems and methods provide for a BAR seizure monitor to excite and listen to the acoustic modes in the entire head (e.g., brain and skull together) by putting a small wearable or implantable transducer over the head. The transducer may be small and provide high spatial bandwidth to excite as many modes as possible. In some embodiments, only one transducer may be sufficient to detect a seizure. In some embodiments, for having more local readings, more transducers can be populated over the head.

In a non-limiting example, a single nerve fiber during electrical activity (or action potential) experiences swelling with a displacement of about 5-10 nm, and a swelling pressure about half a pascal. The frequency of the generated displacement centers around a few kHz. A seizure is expected to result from many firings, and hence is predicted to have a larger displacement, from a larger source, and generate a stronger pressure. This is a low frequency volume change at the seizure site, which will perturb the acoustic modes of the brain that are continuously being monitored by the device. These perturbations are registered at different frequencies, giving in turn enough information to (a) detect the seizure and (b) localize the seizure site.

In some embodiments, this device can be combined with electroencephalogram (EEG) readings and/or other functional imaging techniques such as fMRI, fNIRs, as well as functional optoacoustic/thermoacoustic imaging to enhance the reliability, robustness, accuracy, specificity of detection and localization. In some embodiments, this technology can be combined with Focused Ultrasound (FUS) to detect, localize and suppress the seizure seconds before it leads to any serious complications. Once the seizure is localized with a millimeter resolution, an array of ultrasonic transducers at high frequencies (e.g., 0.5-1 MHz) can be used to suppress the action potential firings, and hence blunt the seizure. Ultrasound energy has been shown to have reversible inhibitory effects, through macroscopic temperature elevation in the brain.

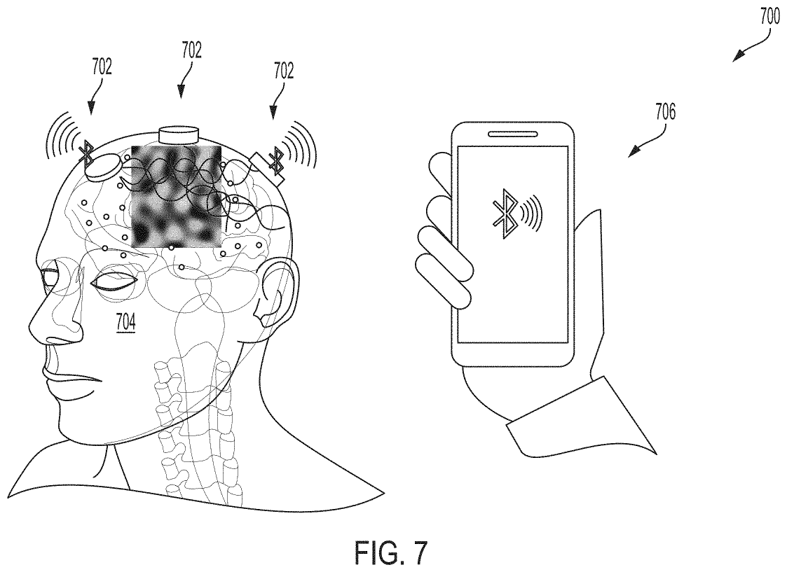

FIG. 7 shows an illustrative example 700 of a BAR seizure monitor, in accordance with some embodiments of the technology described herein. In FIG. 7, acoustic transducers 702 may set up a reverberant field (also known as chaotic standing waves) in the brain 704. The standing waves are a superposition of acoustic resonances of the brain. A seizure creates a local low frequency effect that modulates the behavior of the resonances. The received waveforms may be wirelessly transmitted to a hub 706 like APPLE WATCH or IPHONE via BLUETOOTH.

In some embodiments, a method, a system, and/or a device for a brain acoustic resonance seizure monitor transmits acoustic signals to the brain using one or more transducers. The transducers receives data acquired from the brain, including information related to standing waves, distribution of acoustic modes, frequency response, and/or impulse/transient response. In some embodiments, the same transducer(s) transmit the acoustic signals to the brain and receive the data acquired from the brain. In some embodiments, the transducer(s) used for transmitting the acoustic signals to the brain are different from the transducer(s) used for receiving the data acquired from the brain.

The seizure is detected from the acquired data. For example, detecting the seizure may include assessing changes in amplitude, bandwidth, and/or frequency of the standing waves. Optionally, in addition to detecting the seizure, a location of the seizure site may be determined based on the standing waves. Additionally or alternatively, the acquired data may be transmitted to an external device with a processor to determine the seizure. For example, the seizure may be detected at the external device using a statistical model (e.g., as described with respect to FIGS. 15-16 or another suitable statistical model) that receives at least a portion of the acquired data as input and outputs an indication of seizure or related information suitable for determining the seizure. The device for the brain acoustic resonance seizure monitor may be wearable by the person, implantable within a skull of the person, and/or portable in nature. In some embodiments, the main differences between the BAR for ICP and the BAR for seizure may lie in the inference algorithm, location and population, and/or center frequency of the BAR sensors. In some embodiments, the same device based on BAR may be used to measure ICP and detect seizures.

ICP, Seizure, ICP Distribution, Tumor Detection, and Seizure Localization Algorithms

In some embodiments, the received waveforms (e.g., resonances in the BAR or amplitude and phase of the transmitted waveform in the skull Lamb wave) are processed via a model-based machine learning algorithm. For example, a physical-acoustics model of the patient's head may be constructed and learned through a suitable machine learning technique using the patient's brain under normal conditions. This model can then be used to infer the brain conditions at later times. The same model may be further combined with techniques such as reinforcement learning for continuously learning and adapting to patient's normal and abnormal brain activities.

A machine learning algorithm may be employed in the form of a classification or regression algorithm, which may include one or more sub-components such as convolutional neural networks, recurrent neural networks such as LSTMs and GRUs, linear SVMs, radial basis function SVMs, logistic regression, and various techniques from unsupervised learning such as variational autoencoders (VAE), generative adversarial networks (GANs) which are used to extract relevant features from the raw input data. In some embodiments, the described technology is patient-specific, where computations and model-based learning are implemented by using the patient's head MR or CT scan. The medical images are processed and fed into an acoustic solver, which is then used to train the model-based machine learning algorithm.

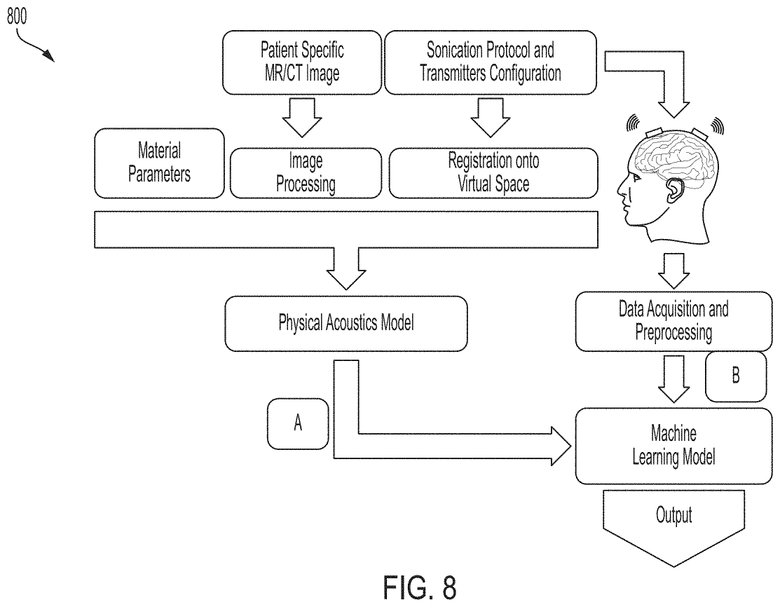

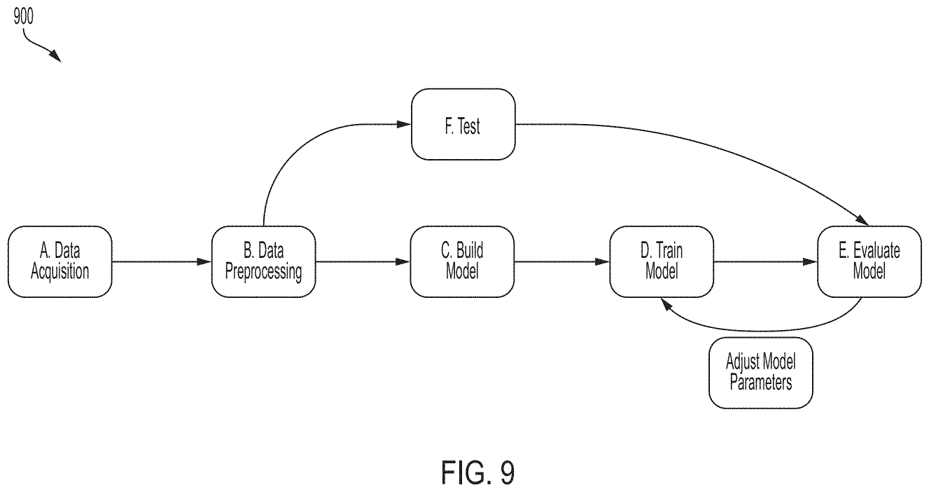

FIG. 8 shows an overview of an illustrative algorithm 800 for determining the intracranial pressure across the brain, its distribution, presence of seizure, location of seizure site, or other indicators of brain functions or conditions. The inputs to the model include the patient specific MR/CT data, sonication protocol and transducers' configuration (e.g., spatial arrangement), as well as material properties such as mechanical and electrical properties, e.g., speed of sounds, density, elasticity, etc. These inputs, after some computer-processing, are fed into a physical acoustics model (such as linear/nonlinear acoustics, electrodynamics, nonlinear continuum, etc.). Nodes A and B represent the outputs of the physical model and the acquired data, which could be in several forms, including but not limited to the frequency response, impulse/transient response, or distribution of acoustic modes. Both A and B are fed into a statistical model or a machine learning model. The final output can be the intracranial pressure across the brain, its distribution, presence of seizure, location of seizure site, or other indicators of brain functions or conditions. Exemplary steps 900 often undertaken to construct and deploy such algorithms are shown in FIG. 9, including data acquisition, data preprocessing, building a model, training the model, evaluating the model, testing, and adjusting model parameters. FIG. 10 shows exemplary input data 1050 from a source 1000, e.g., a patient's head, for BAR-based ICP estimation. For example, this input data may be provided to algorithm 800 for determining the intracranial pressure across the brain.

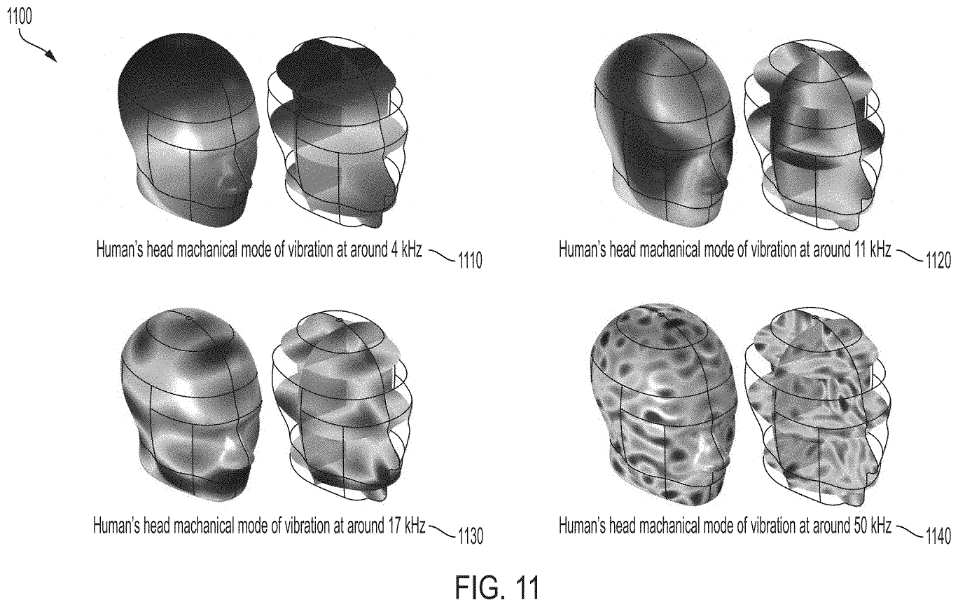

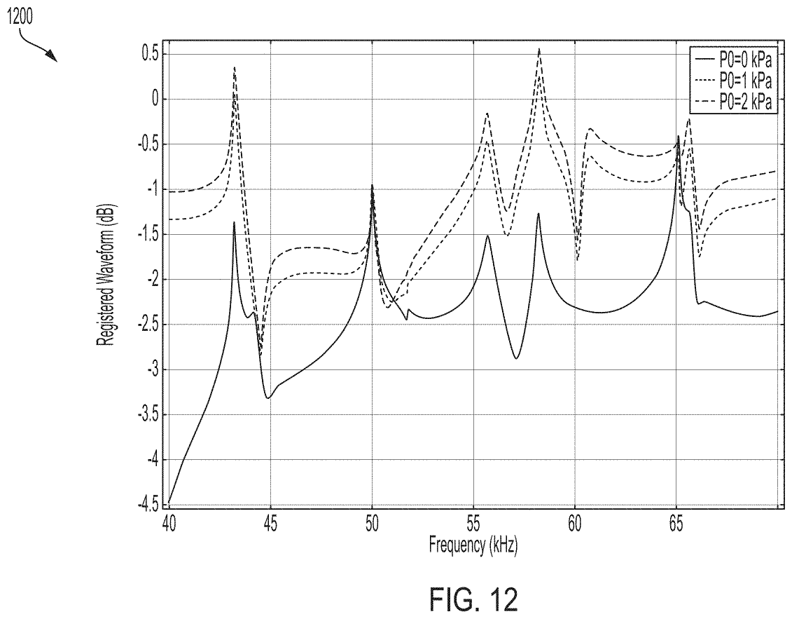

With respect to BAR for ICP and BAR for seizure aspects described herein, examples 1100 of the acoustic resonances in the skull are shown at a selection of frequencies in FIG. 11, including 4 kHz (1110), 11 kHz (1120), 17 kHz (1130), and 50 kHz (1140). The distribution of these resonances is chaotic (or stochastic). In the BAR method, the device collectively excites and listens to these modes using transducers with wide spatial and temporal bandwidths. Any structural changes in the brain such as build-up of the intracranial pressure or local changes in tissue (such as pressure, deformation, volume change, etc.) at the seizure site lead to perturbations of these modes, providing a unique texture in the registered echoes. Using a model-based machine learning algorithm, these changes can be distinguished and quantified. FIG. 12 shows an example 1200 of the response of a sensor over the skull at different intracranial pressures and frequencies. Similarly, using a model-based machine learning algorithm, these changes can be distinguished and quantified.

In some embodiments, a method, a system, and/or a device for tumor detection transmits acoustic signals to the brain and/or skull using one or more transducers. The transducers receives data acquired from the brain and/or skull, including information related to standing waves, guided waves, distribution of acoustic modes, frequency response, and/or impulse/transient response. In some embodiments, the same transducer(s) transmit the acoustic signals to the brain and receive the data acquired from the brain. In some embodiments, the transducer(s) used for transmitting the acoustic signals to the brain are different from the transducer(s) used for receiving the data acquired from the brain.

The presence of a tumor in the brain is detected from the acquired data, e.g., similar to the data shown in FIG. 10. For example, detecting the presence of the tumor may include assessing changes in amplitude, bandwidth, and/or frequency of the standing waves and/or guided waves. Optionally, in addition to detecting the presence of the tumor, a location of the tumor may be determined based on the acquired data. Additionally or alternatively, the acquired data may be transmitted to an external device with a processor to detect the presence of the tumor. For example, the tumor may be detected at the external device using a statistical model (e.g., as described with respect to FIGS. 15-16 or another suitable statistical model) that receives at least a portion of the acquired data as input and outputs an indication regarding the presence of the tumor or related information suitable for determining whether a tumor is present in the brain. The device for tumor detection may be wearable by the person, implantable within a skull of the person, and/or portable in nature.

In some embodiments, a method, a system, and/or a device for mapping distribution of intracranial pressure transmits acoustic signals to the brain using one or more transducers to induce excitation of a plurality of acoustic modes. The transducers receives data acquired from the brain, including information related to standing waves, distribution of acoustic modes, frequency response, and/or impulse/transient response. In some embodiments, the same transducer(s) transmit the acoustic signals to the brain and receive the data acquired from the brain. In some embodiments, the transducer(s) used for transmitting the acoustic signals to the brain are different from the transducer(s) used for receiving the data acquired from the brain.

The distribution of intracranial pressure is determined from the acquired data. For example, determining the distribution of intracranial pressure may include providing the acquired data, e.g., as shown in FIG. 10, to a statistical model or a machine learning model (e.g., as described with respect to FIGS. 15-16 or another suitable statistical model) trained to predict the distribution of intracranial pressure. Additionally or alternatively, the acquired data may be transmitted to an external device with a processor to determine the distribution of intracranial pressure. For example, the distribution of intracranial pressure may be detected at the external device, using the statistical model or machine learning described above, which receives at least a portion of the acquired data as input and outputs a distribution of intracranial pressure or related information suitable for determining the distribution of intracranial pressure. The device for mapping distribution of intracranial pressure may be wearable by the person, implantable within a skull of the person, and/or portable in nature.

In some embodiments, a method, a system, and/or a device for seizure localization transmits acoustic signals to the brain using one or more transducers to induce excitation of a plurality of acoustic modes. The transducers receives data acquired from the brain, including information related to standing waves, distribution of acoustic modes, frequency response, and/or impulse/transient response. In some embodiments, the same transducer(s) transmit the acoustic signals to the brain and receive the data acquired from the brain. In some embodiments, the transducer(s) used for transmitting the acoustic signals to the brain are different from the transducer(s) used for receiving the data acquired from the brain.

The location of the seizure site is determined from the acquired data. For example, determining the location of the seizure site may include providing the acquired data to a statistical model or a machine learning model (e.g., as described with respect to FIGS. 15-16 or another suitable statistical model) trained to predict the location of the seizure site. In some embodiments, in the case of BAR for detecting a seizure, the superposition of resonances sets up reverberation in the brain, which along with coupling into the skull can provide a unique texture into the pressure waves, and thus enable localizations of the source of a seizure to a few millimeters using a sparse configuration of sources. Field reverberation leads to several interrogations (passages) of the acoustic waves over each point in the brain, allowing to register its signature as a function of time or frequency. This can also lead to significant reduction in the number of sensors, in contrary to the conventional wisdom where a large number of sensors is often required for a high-resolution acoustic localization. Additionally or alternatively, the acquired data may be transmitted to an external device with a processor to determine the location of the seizure site. For example, the location of the seizure site may be detected at the external device, using the statistical model or machine learning described above, which receives at least a portion of the acquired data as input and outputs a location of the seizure site or related information suitable for determining the location of the seizure site. The device for seizure localization may be wearable by the person, implantable within a skull of the person, and/or portable in nature.

The sensors, systems and methods described herein can be used to monitor and/or treat epilepsy or brain tumors as described, but the inventions are not so limited. The sensors, systems and methods can be used to monitor and/or treat general brain function and/or other brain conditions, including localizing of source of ICP or seizure or mapping the distribution of ICP, but the inventions are not so limited.

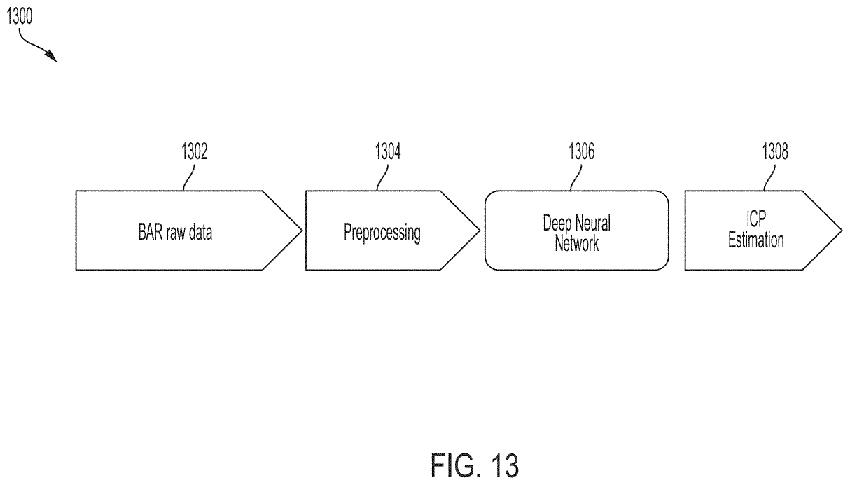

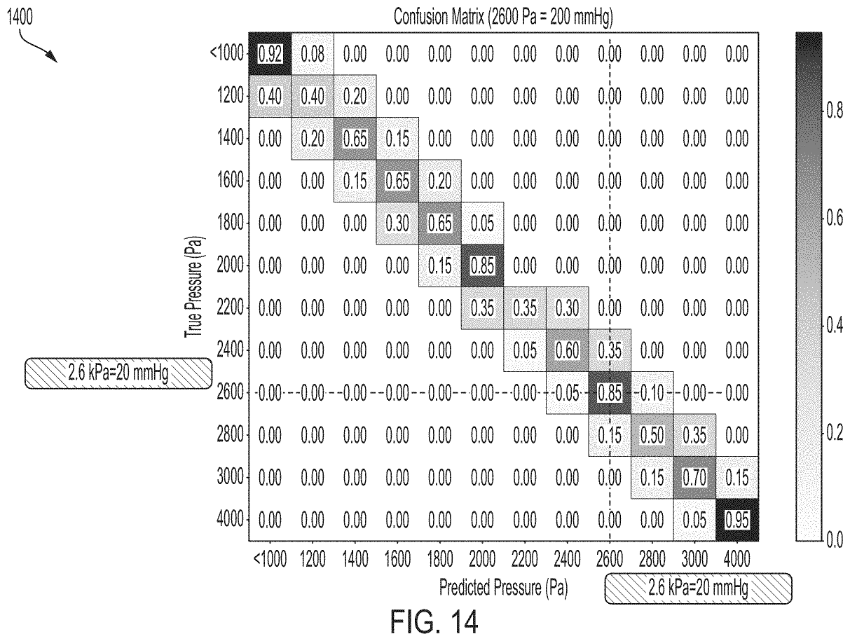

FIG. 13 shows an illustrative flow diagram 1300 for a process for ICP estimation, in accordance with some embodiments of the technology described herein. At step 1302, raw data is received from acoustic resonances in the skull at a selection of one or more frequencies, e.g., as shown in FIG. 11, including 4 kHz, 11 kHz, 17 kHz, 50 kHz, and/or another suitable frequency. At step 1304, this training data is preprocessed for input into a statistical model, e.g., a deep neural network, a statistical model as described with respect to FIGS. 15-16, or another suitable statistical model. For example, the data may be normalized, sanitized, or otherwise made uniform for input to the statistical model. Any structural changes in the brain such as build-up of the intracranial pressure or local changes in tissue (such as pressure, deformation, volume change, etc.) at the seizure site lead to perturbations of the modes, e.g., as shown in FIG. 11, providing a unique texture in the registered echoes. Using a model-based machine learning algorithm, these changes can be distinguished and quantified. Accordingly, at step 1306, the statistical model is trained on the preprocessed training data from step 1304 to predict intracranial pressure or another suitable indication described herein. At step 1308, the statistical model is used to predict intracranial pressure of a person using data acquired from the brain. FIG. 14 shows an illustration 1400 of the performance of an exemplary trained statistical model on test data via a confusion matrix, in accordance with some embodiments of the technology described herein. The diagonal pattern indicates that the statistical model recognizes and predicts the correct intracranial pressure (and that the error is limited to within the neighboring points).

In some embodiments, the systems and methods described herein employ a statistical model for classification, which may include one or more sub-components such as convolutional neural networks, recurrent neural networks such as LSTMs and GRUs, linear SVMs, radial basis function SVMs, logistic regression, and various techniques from unsupervised learning such as variational autoencoders (VAE), generative adversarial networks (GANs) which are used to extract relevant features from the raw input data. Deep neural networks have been the center of attention, for machine learning, due to superior performance, when presented with large datasets. Theoretically, they are capable of learning any functional form, usually a mapping f:Rⁿ→R^{m}, when designed with enough complexity. Although useful for learning from smaller datasets with unknown dynamics and distribution, this flexibility can lead to severe overfitting.

FIG. 15 shows an exemplary arrangement 1500 for the statistical model based on one-shot or few-shot learning. The arrangement includes Siamese neural networks, which can prevent or mitigate overfitting by projecting the data onto a low dimensional representation which encodes only the abstract relative distance between samples. Hence, to train this model, each data point is evaluated against all other data points from the same and different classes. This leads to a quadratic increase in number of input samples. Siamese networks, like the one shown in FIG. 15, are neural networks containing two or more identical subnetwork components. Not only is the architecture of the subnetworks identical, but the weights are shared among them as well for the network. Such networks can learn useful data descriptors that can be further used to compare between the inputs of the respective subnetworks. Input data may include numerical data (e.g. with subnetworks formed by fully-connected layers), image data (e.g., with CNNs as subnetworks), and/or sequential data such as sentences or time signals (e.g., with recurrent neural networks (RNNs) as subnetworks).

In FIG. 15, the illustrative deep convolutional neural network (CNN) projects sensor recordings onto an eight-dimensional feature space in which Euclidean distance represents the difference in pursue. To train this model, a Siamese regime is used to help group the representation vectors belonging to the same pressure together while pushing the ones for different values far from each other. In addition, a class value may be assigned to any of these clusters using a multi-class classifier. The encoder CNNs 1506 and 1508 are made deeper using fractional max-pooling (FMP). Deeper and narrower neural networks have shown better generalization characteristics than wider and shallower ones when the number of parameters is equal. FMP also adds stochasticity and makes the model variational, which makes it less prone to overfitting. Further information on FMP may be found in Benjamin Graham, “Fractional Max-Pooling,” arXiv:1412.6071, May 2015, which is incorporated herein by reference in its entirety. The fully connected layer (FC) towards the end of the CNN architecture operates on a flattened input where each input is connected to all neurons. FC layers can be used to optimize objectives such as class scores. In some embodiments, in the problem at hand, confusing the nearby values may be less costly. Consequently, this information may be incorporated in the objective function by convolving each label with a Gaussian window, before computing the conditional entropy, as the objective function.

In FIG. 15, X1 (1502) and X2 (1504) are the input raw data, e.g., as shown in FIG. 10. In some embodiments, in addition to the raw data being used as is to provide input, any post-processed data such as spectral data (i.e., Fourier transformed raw data), filtered data, windowed data, amplitude of the spectral data, locations of the peaks of the spectral data, bandwidths around the peaks of the spectral data, etc., can be used independently or collectively together to train the machine learning algorithm. H1 (1510) and H2 (1512) are the “encoded” (or latent or hidden) representations of the inputs. H1 and H2 are independently fed into Multiclass Pursue Classifiers 1516 and 1518, respectively. The respective classifiers classify H1 and H2 according to the corresponding “labels,” which here are the pressure (ICP) values. In some embodiments, the labels can be pressure (ICP) values, the distribution of ICP values, the occurrence of a seizure, the location of a seizure, the location of a tumor, or a combination thereof. The modulus of the difference 1514, |H1−H2|, is fed into yet another classifier 1520, e.g., a binary similarity classifier, where the labels are either 1 (e.g., if X1 and X2 correspond to the same pressure value) or 0 (e.g., if X1 and X2 correspond to different pressure values). The algorithm learns the model by optimizing for the parameters that minimize all the outputs. In some embodiments, the optimization function may be defined as a weighted sum of all the outputs.

FIG. 16 shows a convolutional neural network 1600 that may be used to implement a classification algorithm, in accordance with some embodiments of the technology described herein. The statistical model described herein may include the convolutional neural network 1600, and additionally or alternatively another type of network, suitable for detecting and/or predicting whether the brain is exhibiting or will exhibit a symptom of a neurological disorder. For example, convolutional neural network 1600 may be used to detect and/or predict a seizure in the brain. As shown, the convolutional neural network comprises an input layer 1604 configured to receive information about the input 1602 (e.g., a tensor), an output layer 1608 configured to provide the output (e.g., classifications in an n-dimensional representation space), and a plurality of hidden layers 1606 connected between the input layer 1604 and the output layer 1608. The plurality of hidden layers 1606 include convolution and pooling layers 1610 and fully connected layers 1612.

The input layer 1604 may be followed by one or more convolution and pooling layers 1610. A convolutional layer may comprise a set of filters that are spatially smaller (e.g., have a smaller width and/or height) than the input to the convolutional layer (e.g., the input 1602). Each of the filters may be convolved with the input to the convolutional layer to produce an activation map (e.g., a 2-dimensional activation map) indicative of the responses of that filter at every spatial position. The convolutional layer may be followed by a pooling layer that down-samples the output of a convolutional layer to reduce its dimensions. The pooling layer may use any of a variety of pooling techniques such as max pooling and/or global average pooling. In some embodiments, the down-sampling may be performed by the convolution layer itself (e.g., without a pooling layer) using striding.

The convolution and pooling layers 1610 may be followed by fully connected layers 1612. The fully connected layers 1612 may comprise one or more layers each with one or more neurons that receives an input from a previous layer (e.g., a convolutional or pooling layer) and provides an output to a subsequent layer (e.g., the output layer 1608). The fully connected layers 1612 may be described as “dense” because each of the neurons in a given layer may receive an input from each neuron in a previous layer and provide an output to each neuron in a subsequent layer. The fully connected layers 1612 may be followed by an output layer 1608 that provides the output of the convolutional neural network. The output may be, for example, an indication of which class, from a set of classes, the input 1602 (or any portion of the input 1602) belongs to. The convolutional neural network may be trained using a stochastic gradient descent type algorithm or another suitable algorithm. The convolutional neural network may continue to be trained until the accuracy on a validation set (e.g., a held out portion from the training data) saturates or using any other suitable criterion or criteria.

It should be appreciated that the convolutional neural network shown in FIG. 16 is only one example implementation and that other implementations may be employed. For example, one or more layers may be added to or removed from the convolutional neural network shown in FIG. 16. Additional example layers that may be added to the convolutional neural network include: a pad layer, a concatenate layer, and an upscale layer. An upscale layer may be configured to upsample the input to the layer. An ReLU layer may be configured to apply a rectifier (sometimes referred to as a ramp function) as a transfer function to the input. A pad layer may be configured to change the size of the input to the layer by padding one or more dimensions of the input. A concatenate layer may be configured to combine multiple inputs (e.g., combine inputs from multiple layers) into a single output. As another example, in some embodiments, one or more convolutional, transpose convolutional, pooling, unpooling layers, and/or batch normalization may be included in the convolutional neural network. As yet another example, the architecture may include one or more layers to perform a nonlinear transformation between pairs of adjacent layers. The non-linear transformation may be a rectified linear unit (ReLU) transformation, a sigmoid, and/or any other suitable type of non-linear transformation, as aspects of the technology described herein are not limited in this respect.

Any suitable optimization technique may be used for estimating neural network parameters from training data. For example, one or more of the following optimization techniques may be used: stochastic gradient descent (SGD), mini-batch gradient descent, momentum SGD, Nesterov accelerated gradient, Adagrad, Adadelta, RMSprop, Adaptive Moment Estimation (Adam), AdaMax, Nesterov-accelerated Adaptive Moment Estimation (Nadam), AMSGrad.

Convolutional neural networks may be employed to perform any of a variety of functions described herein. It should be appreciated that more than one convolutional neural network may be employed to make predictions in some embodiments.

### Example Computer Architecture

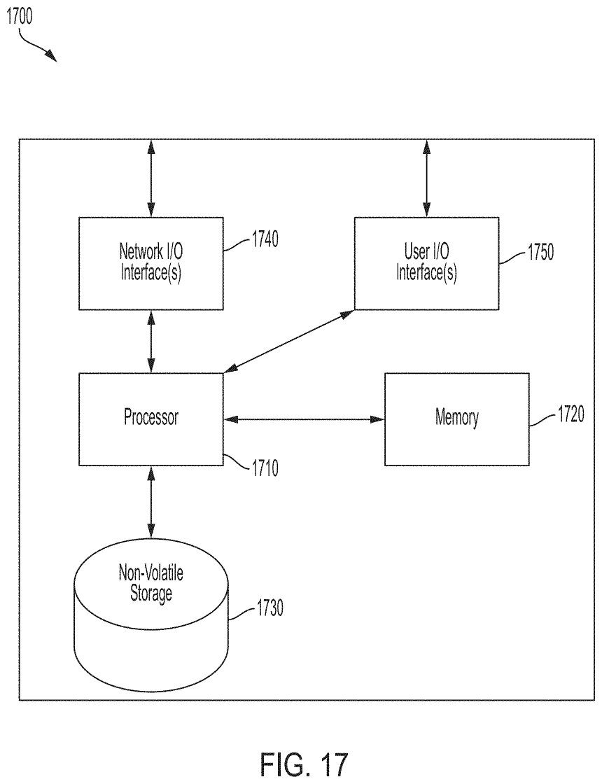

An illustrative implementation of a computer system 1700 that may be used in connection with any of the embodiments of the technology described herein is shown in FIG. 17. The computer system 1700 includes one or more processors 1710 and one or more articles of manufacture that comprise non-transitory computer-readable storage media (e.g., memory 1720 and one or more non-volatile storage media 1730). The processor 1710 may control writing data to and reading data from the memory 1720 and the non-volatile storage device 1730 in any suitable manner, as the aspects of the technology described herein are not limited in this respect. To perform any of the functionality described herein, the processor 1710 may execute one or more processor-executable instructions stored in one or more non-transitory computer-readable storage media (e.g., the memory 1720), which may serve as non-transitory computer-readable storage media storing processor-executable instructions for execution by the processor 1710.

Computing device 1700 may also include a network input/output (I/O) interface 1740 via which the computing device may communicate with other computing devices (e.g., over a network), and may also include one or more user I/O interfaces 1750, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The embodiments described herein can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described herein can be generically considered as one or more controllers that control the functions discussed herein. The one or more controllers can be implemented in numerous ways, such as with dedicated hardware, or with general purpose hardware (e.g., one or more processors) that is programmed using microcode or software to perform the functions recited herein.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the functions discussed herein of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the functions discussed herein, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The terms “program” or “software” are used herein in a generic sense to refer to any type of computer code or set of processor-executable instructions that can be employed to program a computer or other processor to implement various aspects of embodiments as discussed herein. Additionally, it should be appreciated that according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Also, various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing,” “involving”, and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

While some aspects and/or embodiments described herein are described with respect to intracranial pressure or epilepsy-related applications, these aspects and/or embodiments may be equally applicable to monitoring and/or treating symptoms for any suitable neurological disorder. Any limitations of the embodiments described herein are limitations only of those embodiments, and are not limitations of any other embodiments described herein.

What is claimed is:

1. A method comprising:
transmitting to a brain of a patient, with at least one transducer, acoustic signals;
receiving from the brain, with the at least one transducer, data acquired from the brain including information related to standing waves, distribution of acoustic modes, frequency response, and/or impulse/transient response; and
detecting, from the acquired data, a seizure of the person.

2. The method of claim 1, wherein detecting the seizure includes assessing changes in amplitude, bandwidth, and/or frequency of the standing waves.

3. The method of claim 1, further including transmitting, to an external device with a processor for detecting the seizure, the acquired data.

4. The method of claim 1, further including determining a location of a seizure site based on the standing waves.

5. The method of claim 1, wherein the at least one transducer includes a first transducer for transmitting the acoustic signals and receiving the acquired data.

6. The method of claim 1, wherein the at least one transducer includes a first transducer for transmitting the acoustic signals and a second transducer for receiving the acquired data.

7. A device comprising:
at least one transducer that transmits to the brain of a person acoustic signals and receives data acquired from the brain including information related to standing waves, distribution of acoustic modes, frequency response, and/or impulse/transient response, for detecting, based on the acquired data, a seizure of the person.

8. The device as claimed in claim 7, wherein the device is wearable by the person.

9. The device as claimed in claim 7, wherein the device is implantable within the skull of the person.

10. The device as claimed in claim 7, wherein the device is portable.

11. The device as claimed in claim 7, wherein the at least one transducer includes a first transducer for transmitting the acoustic signals and receiving the acquired data.

12. The device as claimed in claim 7, wherein the at least one transducer includes a first transducer for transmitting the acoustic signals and a second transducer for receiving the acquired data.

13. At least one non-transitory computer-readable storage medium storing processor-executable instructions that, when executed by at least one computer hardware processor, cause the at least one computer hardware processor to perform the acts of:
transmitting to a brain of a patient, with at least one transducer, acoustic signals;
receiving, from the brain, with the at least one transducer, data acquired from the brain including information related to standing waves, distribution of acoustic modes, frequency response, and/or impulse/transient response; and
detecting, from the acquired data, a seizure of the person.

14. The computer-readable storage medium of claim 13, wherein detecting the seizure includes providing the acquired data to a machine learning model trained to predict the seizure of the person.

15. The computer-readable storage medium of claim 13, wherein the at least one transducer includes a first transducer for transmitting the acoustic signals and receiving the acquired data.

16. The computer-readable storage medium of claim 13, wherein the at least one transducer includes a first transducer for transmitting the acoustic signals and a second transducer for receiving the acquired data.
